(19) United States
(12) Patent Application Publication
(10) Pub. No.: US 2004/0006127 A1
(43) Pub. Date: Jan. 8, 2004

(54) Activation of the cystic fibrosis transmembrane conductance regulator chloride channel

(76) Inventors: David Noel Sheppard, Bristol (GB); Zhiwei Cai, Bristol (GB)

Correspondence Address: BOZICEVIC, FIELD & FRANCIS LLP, 200 MIDDLEFIELD RD, SUITE 200, MENLO PARK, CA, 94025, US

(21) Appl. No.: 10/332,972

(22) PCT Filed: Jul. 12, 2001
(86) PCT No.: PCT/GB01/03151

(30) Foreign Application Priority Data
Jul. 13, 2000 (GB) 0017083.7

(51) Int. Cl. A61K 31/353

(52) U.S. Cl. 514/454

(57) ABSTRACT

Fluorescein and derivatives have use in the treatment of a disease of condition of a living animal body, including human, which disease is responsive to the activation of the cystic fibrosis transmembrane conductance regulator chloride channels, for instance cystic fibrosis, disseminated brocheiectasis, pulmonary infections, chronic pancreatitis, male infertility and long QT syndrome.

The present invention relates to the activation of the cystic fibrosis transmembrane conductance regulator (CFTR) chloride (Cl⁻) channel. More particularly, t relates to members of a defined class of chemical compounds as activators of the CFTR Cl⁻ channel and the use of these agents in the treatment of diseases caused by the dysfunction of the CFTR Cl⁻ channel.

CFTR (1) forms a Cl⁻ channel with complex regulation (2,3). It is predominantly expressed in the apical membrane of epithelia, where it provides a pathway for the movement of Cl⁻ ions and a key point at which to regulate the rate of transepithelial salt and water movement (4).

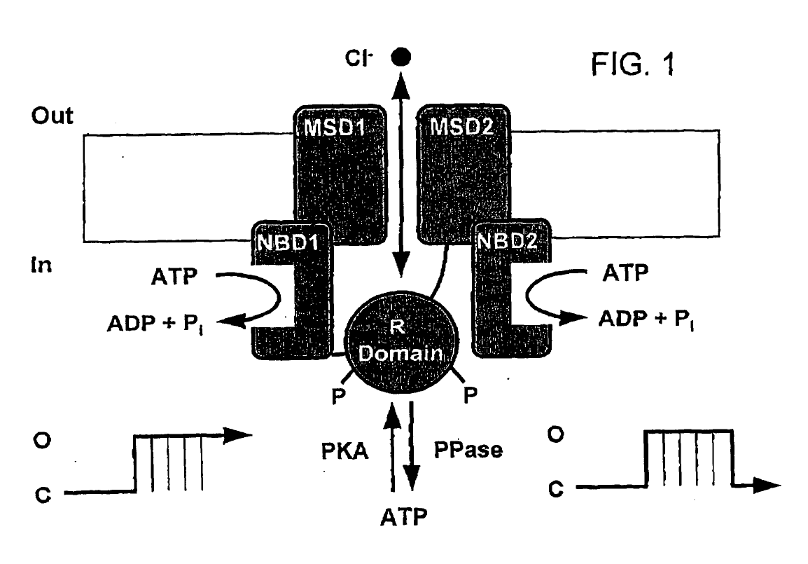

FIG. 1: Regulation of the CFTR Cl⁻ Channel.

The domain structure of the cystic fibrosis transmembrane conductance regulator (CFTR) showing the regulation of channel gating is illustrated diagrammatically in FIG. 1. Schematic representations of channel gating are shown below the model: C, closed state; O, open state. Abbreviations: MSD, membrane-spanning domain; NBD, nucleotide-binding domain; P, phosphorylation of the R domain; PKA, cAMP-dependent protein kinase; PPase, phosphatase; R, regulatory domain; In, intracellular; Out, exracellular. The white box represents the cell membrane.

CFTR is composed of five domains: two membrane-spanning domains (MSDs), two nucleotide-binding domains' (NBDs), and a regulatory (R) domain (1). The MSDs contribute to the formation of the Cl⁻-selective pore, while the NBDs and R domain control channel activity (2,3). The activation of the cAMP-dependent protein kinase (PKA) causes the phosphorylation of multiple serine residues within the R domain. Once the R domain is phosphorylated, channel gating is controlled by a cycle of ATP hydrolysis at the NBDs.

Dysfunction of CFTR is associated with a wide spectrum of disease. Mutations which, in general, abolish the function of CFTR cause the genetic disease cystic fibrosis (CF; 4). However, some forms of male infertility, disseminated bronchi ctasis, and chronic pancreatitis are also caused by mutations, which, it is thought, preserve partial CFTR function (4). A greater than normal activity of the CFTR Cl⁻ channel is thought to be implicated in certain other diseases, for example polycystic kidney disease and secretory diarrhoea (5,6).

Over 900 disease-causing mutations have been identified in the CFTR gene. Based on studies of wild-type and mutant CFTR Cl⁻ channels, Welsh and Smith (7), identified four general mechanisms that cause a loss of Cl⁻ channel function: mechanisms that disrupt protein production, protein processing, channel regulation and channel conduction. Importantly, many CF-associated mutations have some residual Cl⁻ channel function when expressed in heterologous cells. This Includes mutations that disrupt the biosynthesis of CFTR, such as the most common CF-associated mutation, deletion of a phenylalanine residue at position 508 of the CUR sequence (termed ΔF508; 4). Thus, pharmacological agents that enhance the activity of mutant Cl⁻ channels, present at the apical membrane of epithelia, may be of value in the treatment of CF and other diseases, which are responsive to the activation of CFTR Cl⁻ channels.

The compound genistein, 5,7-dihydrox-3-(4-hydroxyphenyl)-4H-1-benzopyran-4-one, has been found to potentate the activity of PKA-phosphorylated wild-type and ΔF508 CFTR Cl⁻ channels (8). Unfortunately, genistein interacts with numerous other targets within cells at concentrations similar to those that stimulate CFTR.

Nucleoside triphosphates, such as ATP, control the activity of the CFTR Cl⁻ channel. Once phosphorylated by PKA, micromolar concentrations of ATP are required to regulate the opening and dosing of the CFTR Cl⁻ channel. Intracellular ATP also regulates a class of K⁺ channels, termed ATP-sensitive K⁺ channels (K_{ATP} channels; 9). The opening of K⁺ channels in pancreatic β cells, myoctes, and some neurons is coupled to the cytoplasmic concentration of ATP. In contrast to the CFTR Cl⁻ channel, where ATP interacts with the NBDs to control channel gatng. ATP prevents K⁺ flow through the pore of the K_{ATP} channel to inhibit channel activity.

Certain derivatives of the compound fluorescein have been found to modulate the activity of K_{ATP} channels (10). The fluorescein derivatives were found to have two opposite effects. Firstly, they can inhibit K_{ATP} channels. Secondly, they are able to reactivate K channels that have become inactivate (termed “run-down” in the absence of cytoplasmic components required to maintain channel activity.

The present invention is based on the discovery that certain fluorescein derivatives can be used to activate CFTR Cl⁻ channels and, as a consequence, that these compounds have use In the treatment of a disease or condition that is responsive to the activation or stimulation of the CFTR Cl⁻ channel, for instance CF, disseminated bronchiectasis, pulmonary infections, chronic pancreatitis and the acquired and inherited forms of long QT syndrome.

Accordingly, the present invention provides use of a compound of the formula I

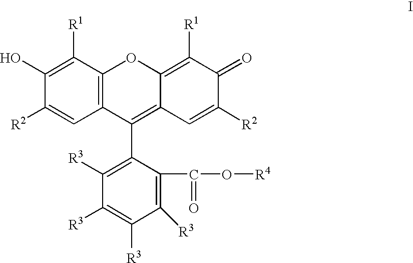

wherein R¹, R² and R³ may each be the same or a different group selected from H, 1 to 6C alkyl and halo and R⁴ is a group selected from H and 1 to 6C alkyl, and pharmaceutically-acceptable salts thereof, in the manufacture of a medicament for the treatment of a disease or medical condition of a living animal body, including a human, which disease or condition is responsive to the activation or stimulation of the CFTR Cl⁻ channel.

The invention further provides a method of treating a disease or medical condition of a living animal body. Including a human, which disease or condition is responsive to the activation of the CFTR or channel which method comprises administering to the living animal body a CFTR chloride channel activating amount of a compound of the formula I.

The fluorescein compounds that have been found to be useful in carrying out the present invention have the formula I

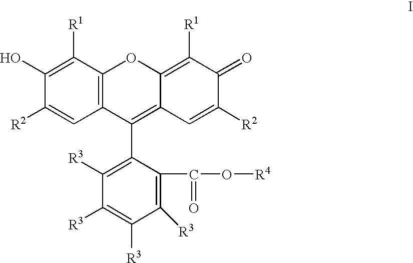

in which R¹, R² and R³ may each be the same or different group selected from H, 1 to 6C alkyl and halo and R⁴ is a group selected from H and 1 to 6C alkyl groups or a pharmaceutically-acceptable salt-forming cationic group. Preferable are compounds in which R¹, R² and R³ is the same or different group selected from H, a 1 to 4C alkyl group or pharmaceutically-acceptable salts of the carboxylic acid when R⁴ is H. More preferable, are compounds of the formula I above in which R¹ is selected from H, Cl, Br and 1, R² is selected from H, Cl, Br and 1, R³ is selected from H, Cl, Br and I and R⁴ is selected from H, methyl and ethyl.

Fluorescein Is a well-known compound. It, and is derivatives as described herein, can be synthesised according to procedures known in the art. The active compound may be administered to the animal body, including human, requiring treatment by any appropriate means. Typically, administration of the active compound, or medicinal preparation containing it, will be by an oral or intravenous route, or in the form of an aerosol or spray for introduction of the active compound into the air passages and lungs of the patient.

As mentioned above, compounds of the formula I have use as pharmaceutically-active ingredients in the treatment of an animal body, including a human suffering from a disease or condition which is responsive to the activation or stimulation of the CFTR Cl⁻ channel. The dosage administered to the animal body in need of therapy will, of course, depend on the actual active compound used, the mode of treatment and the type of treatment required. The active compound may, of course, be administered on its own or in the form of an appropriate medicinal composition containing, for instance, an appropriate pharmaceutically-acceptable carrier or diluent. Other substances may also be present in such medicinal compositions, such as adjuvants and stabilizers the use of which is well known to persons skilled in the art. The active compound may be administered to the animal body, including human, requiring treatment by any appropriate means. Typically, administration of the active compound, or medicinal preparation containing it, will be by an oral or intravenous route or in the form of an aerosol or spray for introduction of the active compound into the air passages or lungs of the patient.

As mentioned earlier, it is believed that the fluorescein derivatives having the formula 1, as defined above, have use in the treatment of a disease or condition that is responsive to the activation or stimulation of the CFTR Cl⁻ channel. Examples of such diseases or conditions include cystic fibrosis, disseminated bronchiectasis, pulmonary infections, chronic pancreatitis, certain forms of male infertility and the acquired and inherited forms of long QT syndrome. In addition, we believe that these fluorescein derivatives may be used to increase fluid secretion into the respiratory airways and, thus, provide a treatment, as expectorant, for a dry irritant unproductive cough. Furthermore, we believe that these fluorescein compounds may be used to increase fluid secretion into the intestines and, thus, provide a treatment, as purgative, to treat constipation.

### EXPERIMENTAL METHODS

Methods

Cell Culture

For electrophysiological experiments, we used mouse mammary epithelial cells (C127 cells) stably expressing wild-type human CFTR or ΔF508, the most common CF-associated mutation (11). C127 cells expressing wild-type CFTR were cultured as previously described (12). C127 cells expressing ΔF508 were cultured at 28° C., to overcome the processing defect of ΔF508 and promote bs delivery to the cell membrane (13). Cells were seeded onto glass coverslips and used within either 48 h (wild-type CFTR) or 1 week (ΔF508).

For iodide efflux experiments, we used Type I Mardin-Darby canine kidney (MDCK) cells. MDCK cells were cultured in MDCK media (a 1:1 mixture of Dulbecco's Modified Eagle Medium (DMEM) and Ham's F-12 nutrient medium supplemented with 10% fetal bovine serum, 100 U ml¹ penicillin, and 100 mg ml⁻streptomycin; all from Life Technologies Ltd, Paisley, UK) at 37° C. in a humidified atmosphere of 5% CO₂. Cells were seeded onto 60 mm plastic culture dishes and used 72-96 h later.

Electrophysiology

CFTR Cl⁻ channels were recorded in excised inside-out membrane patches using an Axopatch 200A patch-clamp amplifier (Axon Instruments Inc., Foster City, USA) and pCLAMP data acquisition and analysis software (version 6.03, Axon Instruments Inc.) as previously described (12,14). The established sign convention was used throughout; currents produced by positive charge moving from intra- to extracellular solutions (anions moving in the opposite direction) are shown as positive currents.

The pipette (extracellular) solution contained (in mM): 140 N-methyl-D-glucamine (NMDG), 140 aspartic acid, 5 CaCl₂, 2 MgSO₄, and 10 N-tris[hydroxymethyl]methyl-2-aminoethanesulphonic acid (Tes), pH 7.3 with Tris ([Cl⁻], 10 mM). The bath (intracellular) solution contained (in mM): 140 NMDG, 3 MgCl₂, 1 ethylene glycol-bis(β-aminoethylether)-N,N,N′,N′-tetraacetic acid caesium salt (CsEGTA), and 10 Tes, pH 7.3 with HCl, ([Cl⁻], 147 mM; [Ca²⁺]_{free}, <10⁻⁸ M). All experiments were conducted at 37° C.

After excision of membrane patches from C127 cells. CFTR Cl⁻ channels were activated by the addition of the catalytic subunit of protein kinase A (PKA; 75 nM) and ATP (1.0 mM) to the intracellular solution within 5 min of patch excision. The ATP concentration was subsequently reduced to 0.3 mM (the EC₅₀ for activation of CFTR Cl⁻ channels by intracellular ATP). PKA was maintained in the intracellular solution for the duration of experiments. Voltage was −50 mV. To investigate the effect of phloxine B on CFTR Cl⁻ channels, we used membrane patches containing large numbers of active channels for time course studies and membrane patches containing five or less active channels for single-channel studies. The number of channels per patch was determined from the maximum number of simultaneous channel openings observed during the course of an experiment, as previously described (12). Because the effects of phloxine B on CFTR Cl⁻ channels were only partially reversible (n 6, data not shown), specific interventions were not bracketed by control periods made with the same concentration of ATP and PKA, but without phloxine B. However, we have previously shown that in the continuous presence of PKA and ATP, run-down of CFTR Cl⁻ channels in excised membrane patches from C127 cells is minimal (12). To investigate whether modulation of CFTR by fluorescein derivatives was voltage dependent, we used a voltage ramp protocol. Macroscopic current-voltage (I-V) relationships were obtained. In the absence and presence of fluorescein derivatives by averaging currents generated by 15-30 ramps of voltage, each of 2 s duration; holding voltage was −50 mV. Basal currents recorded in the absence of PKA and ATP were subtracted from those recorded in the absence and presence of fluorescein derivatives to determine the effect of fluorescein derivatives on CFTR Cl⁻ currents,

CFTR Cl⁻ currents were initially recorded on digital audiotape using a digital tape recorder (Biologic Scientific Instruments, model DTR-1204; lntracel Ltd, Royston, UK) at a bandwidth of 10 kHz. On playback, records were filtered with an eight-pole Bessel filter (Frequency Devices, model 902LPF2: Scensys Ltd, Aylesbury, UK) at a corner frequency of 500 Hz and acquired using a Digidata 1200 interface (Axon Instruments Inc.) and pCLAMP at sampling rates of either 2.5 kHz (time course studies) or 5.0 kHz (single-channel studies).

In time course studies, each point is the average current for a 4 s period with data points collected continuously, no data were collected while solutions were changed. Average current (I) for a specific intervention was determined as the average of all the data points collected during the intervention. The relationship between drug concentration and CFTR inhibition was fitted to the Hill equation:

I_{Drug}/I_{Control}=1/{1+([Drug]/Kᵢ)ⁿ}, (1)

where [Drug] is the concentration of drug, I_{Drug}/I_{Control} is the fractional current at the indicated drug concentration relative to that in the same solution in the absence of added drug, Kᵢ is the drug concentration causing half-maximal inhibition, and n is the slope factor (Hill coefficient). Mean data were fitted to a linear form of equation (1) using linear least-squares regression to yield Kᵢ and n values.

To measure single-channel current amplitude (I), Gaussian distributions were fitted to current amplitude histograms, For open probability (Pₒ) and kinetic analyses, lists of open and closed times were created using a half-amplitude crossing criterion for event detection. Transitions <1 ms in duration were excluded from the analyses. Pₒ was calculated using the equation:

Pₒ=(T₁+T₂+ . . . +T_{N})/(NTₜₒₜ), (2)

where N is the number of channels; Tₜₒₜ is the total time analysed, and T₁ is the time that one or more channels are open, T₂ is the time two or more channels are op n and so on. Burst analysis was performed as described by Carson et al. (15), using membrane patches that contained only a single active channel and a to (the time that separates interburst closures from intraburst closures) of 15 ms. Closures longer than 15 ms were considered to define interburst closures, whereas closures shorter than this time were considered gaps within bursts. The mean interburst duration (T_{c}) was calculated using the equation:

Pₒ=T_{b}/(T_{b}+T_{c}), (3)

where T_{b}=a (mean burst duration×the open probability within a burst). Mean burst duration, and open probability within a burst were determined directly from experimental data; Pₒ was calculated using equation (2). Only membrane patches that contained a single active channel were used for single-channel kinetic analyses.

Iodide Efflux

Type I MDCK cells (≧90% confluent) were incubated for 1 h in a loading buffer containing (mM): 136 Nal, 3 KNO₃, 2 Ca(NO₃)₂, 11 glucose, and 20 Hepes, adjusted to pH 7.4 with NaOH. To remove extracellular iodide, cells were thoroughly washed with efflux buffer (136 mM NaNO₃, replacing 136 mM Nal in the loading buffer) and then equilibrated in 2.5 ml efflux buffer for 1 min. The efflux buffer was changed at 1 min intervals over the duration of the experiment, which typically lasted 18 min. Four minutes after anion substitution, cells were exposed to agonists for 4 min. The amount of iodide in each 2.5 ml sample of efflux buffer was determined using an iodide-selective electrode (Russell pH Ltd. Auchtermuchty, UK). Cells were loaded and experiments performed at room temperature (23° C.).

Reagents

The catalytic subunit of PKA was purchased from Promega Ltd. ATP (disodium salt), bengal rose B, eosin Y, fluorescein, phloxine B, Tes and tetrachlorofluorescein were obtained from Sigma-Aldrich Company Ltd (Poole, UK). All other chemicals were of reagent grade.

Fluorescein derivatives are based on structure I above. For fluorescein, R¹, R², R³, and R⁴ are H. For tetrachlorofluorescein, R¹, R², and R⁴ are H and R³ is Cl. For eosin Y, R¹ and R² are both Br and R³ and R⁴ are both H. For phloxine B, R¹ and R² are both Br, R³ is Cl and R⁴ is H. For bengal rose B, R¹ and R² are both I, R³ is Cl and R⁴ is H.

Stock solutions of fluorescein derivatives were prepared in dimethyl sulphoxide and stored at −20° C. Immediately before use, stock solutions were diluted in intracellular solution to achieve final concentrations. The vehicle did not affect either the activity of CFTR Cl⁻ channels or iodide efflux (16).

Statistics

Results are expressed as mean±SEM of n observations. To compare sets of data, we used Student's t test. Differences were considered statistically significant when P<0.05. All tests were performed using SigmaStat (version 1.03, Jandel Scientific GmbH, Erkrath, Germany).

Results

Phloxine B Modulates the Activity of CFTR Cl⁻ Currents

To examine the effect of phloxine B on wild-type human CFTR, we studied CFTR Cl⁻ currents in excised inside-out membrane patches from C127 cells stably expressing wild-type human CFTR. Addition of phloxine B to the solution bathing the intracellular side of the membrane in the absence of either ATP (n=4) or PKA (n=4) was without effect on the activity of CFTR Cl⁻ channels (data not shown). However, when phloxine B was added to the intracellular solution in the continuous presence of PKA and ATP channel activity was altered.

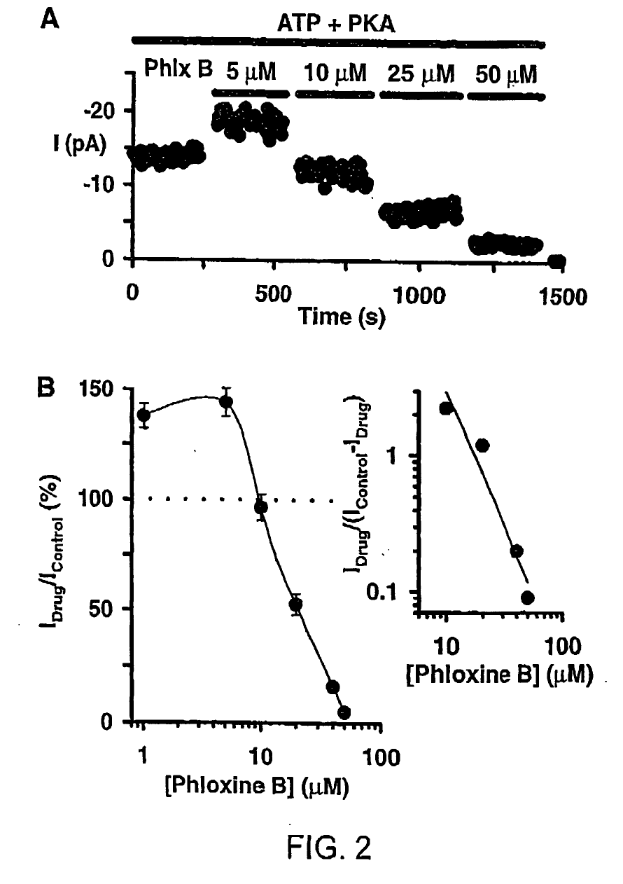

FIG. 2: Phloxine B Modulates the Activity of CFTR

A, time course of CFTR Cl⁻ current in an excised inside-out membrane patch from a C127 cell stably expressing wild-type CFTR. ATP (0.3 mM), PKA (75 nM), and phloxine B (Phlx B; 5-50 μM) were present in the intracellular solution during the times indicated by the bars. Voltage was −50 mV, and there was a Cl⁻ concentration gradient across the membrane (internal [Cl⁻]=147 mM; external [Cl⁻]=10 mM). Each point is the average current for a 4 s period and no data were collected while solutions were changed. For the purpose of illustration, the time course has been inverted so that an upward deflection represents an inward current. B, effect of phloxine B concentration on CFTR Cl⁻ currents. Data are means±SEM; n=4-7 observations at each concentration. Values above the dotted line indicate stimulation of CFTR, whereas values below the line indicate inhibition. Other details as in A. The inset shows a Hill plot of phloxine B inhibition of CFTR. The continuous line is the fit of a first order regression to the data.

FIG. 2 demonstrates that phloxine B modulates the activity of CFTR Cl⁻ currents. Addition of phloxine B (1-5 μM) to the intracellular solution stimulated CFTR Cl⁻ currents. In contrast, higher concentrations of phloxine B (20-50 μM) caused a concentration-dependent decrease in CFTR Cl⁻ current. The relationship between phloxine B concentration and current inhibition was well fitted by the Hill equation with Kᵢ=17 μM and n=2 at −50 mV (FIG. 28 inset). Modulation of CFTR by phloxine B was partially reversible (n=6, data not shown).

In addition to phloxine B, we tested other fluorescein derivatives, including bengal rose B, eosin Y, fluorescein and tetrachlorofluorescein.

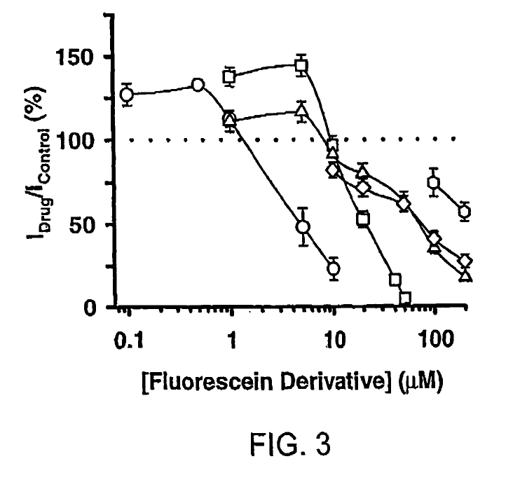

FIG. 3: Concentration-Response Relationships of Fluorescein Derivatives

Data are means±SEM; n=3-9 observations at each concentration. Fluorescein derivatives are indicated by different symbols (bengal rose B, circles; phloxine B, squares; eosin Y, triangles; tetrachlorofluorescein, diamonds; fluorescein, hexagons). Other details as in FIG. 2.

FIG. 3 demonstrates that bengal rose B, eosin Y and phloxine B stimulated and inhibited CFTR Cl⁻ currents, whereas fluorescein and tetrachlorofluorescein only inhibited channel activity. The rank order of potency for CFTR stimulation was bengal rose B (0.1-1.0 μM)>phloxine B (1-5 μM)≧eosin Y(1-5 μM; FIG. 3).

The data presented in FIGS. 2 and 3 indicate that fluorescein derivatives stimulate CFTR Cl⁻ channels over a narrow range of concentrations at −50 mV above which these agents inhibit channel activity. However, analysis of the mechanism of CFTR inhibition by fluorescein derivatives indicates that membrane voltage influences the effect of fluorescein derivatives on the activity of CFTR.

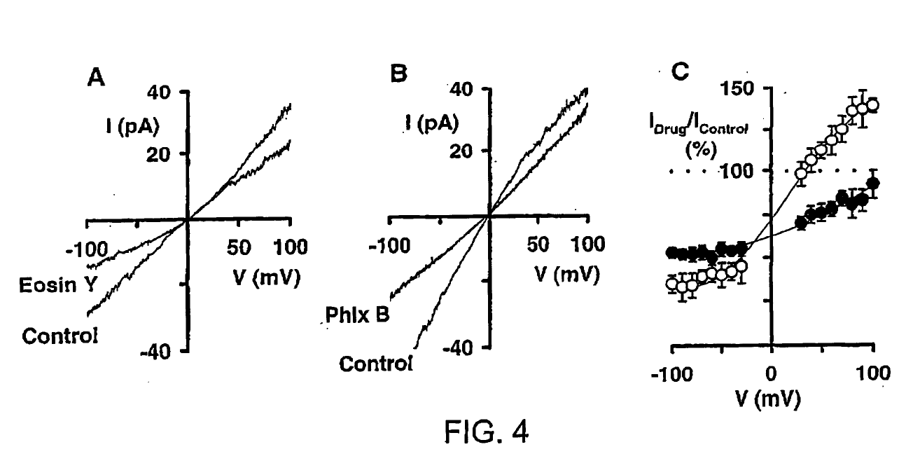

FIG. 4: Effect of Voltage on Eosin Y and Phloxine B Modulation of CFTR Cl⁻ Currents

A and B, I-V relationships of CFTR Cl⁻ currents recorded in the absence and presence of eosin Y (100 μM) and phloxine B (40 μM), respectively, when the membrane patch was bathed in symmetrical 147 mM Cl⁻ solutions. ATP (1 mM) and PKA (76 nM) were continuously present in the intracellular solution. I-V relationships were generated as described in the Methods; holding voltage was −50 mV. C, effect of voltage on the fraction of CFTR Cl⁻ current inhibited by eosin Y (100 μM; open circles) and phloxine B (40 μM; filled circles), respectively. Data are means±SEM (n=4-5) at each voltage. Values above the dotted line indicate stimulation of CFTR, whereas values below the line indicate inhibition. Other details as in A and B.

FIG. 4 demonstrates that eosin Y (100 μM) inhibits channel activity at negative voltages. However, at positive voltages channel activity is stimulated. By contrast, positive voltages relieve channel block by phloxine B (40 μM), but do not stimulate channel activity. Based on these data, we conclude that fluorescein derivatives can stimulate CFTR Cl⁻ channels over a wide range of concentrations.

Mechanism of Phloxine B Stimulation of CFTR

In principal, phloxine B might stimulate the CFTR Cl⁻ channel in one of three ways: first, it might increase the number of active channels present in a membrane patch. Second, it might enhance the amount of current flowing through an open channel. Third, it might increase Pₒ. To discriminate between these different possibilities, we investigated the effect of phloxine B (1 μM) on CFTR Cl⁻ channels using membrane patches that contained ≦5 active channels.

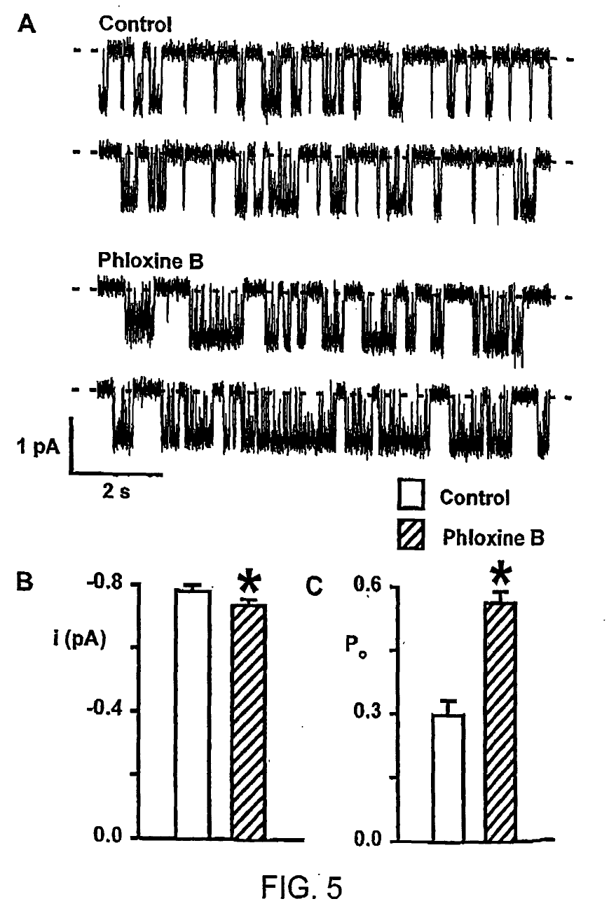

FIG. 5: Effect of Phloxine B (1 μM) on the Single-Channel Activity of CFTR

A, representative recordings show the effect of phloxine B (1 μM) on the activity of a single CFTR Cl⁻ channel. ATP (0.3 mM) and PKA (75 nM) were continuously present in the intracellular solution, Voltage was −50 mV, and there was a Cl⁻ concentration gradient across the membrane (internal [Cl⁻]=147 mM; external [Cl⁻]=10 mM). Dashed lines indicate the closed channel state and downward deflections correspond to channel openings. B and C, effect of phloxine B on i and Pₒ, respectively. Columns and error bars indicate means±SEM; n=10 observations at each concentration. The asterisks indicate values that are significantly different from the control value (P<0.05). Other details as in A.

FIG. 5A shows the effect of phloxine B (1 μM) on the activity of a single CFTR Cl⁻¹ channel following phosphorylation by PKA. Visual inspection of these and other traces (n=10) suggests that phloxine B (1 μM) did not stimulate CFTR by enhancing the number of active channels present. Similarly, phloxine B (1 μM) did not stimulate CFTR by increasing the amount of current flowing through an open channel. On the contrary, the drug caused a small, but significant (P<0.05), reduction in single-channel current amplitude (i; FIG. 5B). Instead, phloxine B (1 μM) altered the gating behaviour of CFTR to cause a large increase in Pₒ (P<0.0001; FIGS. 5A and C). The pattern of gating of wild-type CFTR is characterised by bursts of activity interrupted by brief flickery closures separated by longer closures between bursts (FIG. 5A, top traces). In the presence of phloxine B (1 μM), the gating behaviour of CFTR was characterised by a large increase in the duration of bursts of activity, but no change in the interburst interval (FIG. 5A, bottom traces). We also tested the effect of bengal rose B and eosin Y on the single-channel activity of CFTR. Like, phloxine B, these agents stimulated channel activity by altering the pattern of gating of CFTR and hence, increasing Pₒ (bengal rose B, control Pₒ=0.31+0.06, bengal rose B (1 μM) Pₒ=0.40±0.08, n=4, P<0.05; eosin Y, control Pₒ=0.38±0.02, eosin Y (1 μM) Pₒ=0.43±0.02, n=7, P<0.05).

To determine how phloxine B (1 μM) increased Pₒ we investigated its effect on the gating kinetics of phosphorylated CFTR Cl⁻ channels using membrane patches that contained only a single active channel.

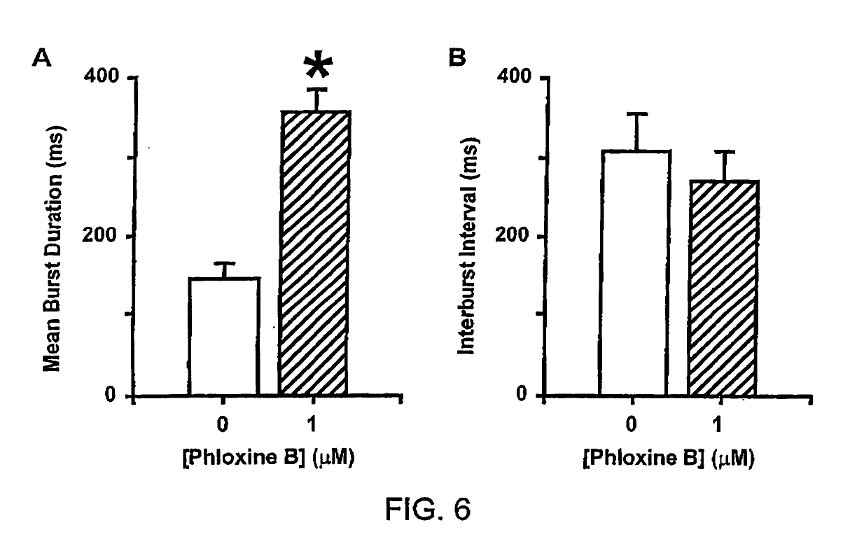

FIG. 6: Burst Duration (A) and Interburst Interval (B) for a Single CFTR Cl⁻ Channel Stimulation by Phloxine B (1 μM)

Columns and error bars indicate means±SEM; n=6 observations at each concentration. Other details as in FIG. 5A. The asterisk indicates a value that is significantly different from the control value (P<0.05).

FIG. 6 shows that phloxine B (1 μM) was without effect on the interburst interval (P>0.05), but significantly increased mean burst duration (P<0.05). These results suggest that phloxine B (1 μM) stimulates CFTR by inhibiting channel closure.

Effect of ATP and ADP on Phloxine B Stimulation of CFTR

The effect of phloxine B (1 μM) on channel gating is reminiscent of that of several activators of the CFTR Cl⁻ channel, including the non-hydrolysable ATP analogue 5′-adenylylimidodlphosphate (AMP-PNP) and genistein (17,18). These agents directly interact with NBD2, which regulates channel closure (2,3). Based on these data, we speculated that phloxine B might compete with ATP for a common binding site on NBD2. To test this hypothesis, we examined the effect of ATP concentration on phloxine B (1 μM) stimulation of CFTR, Cl⁻ channels.

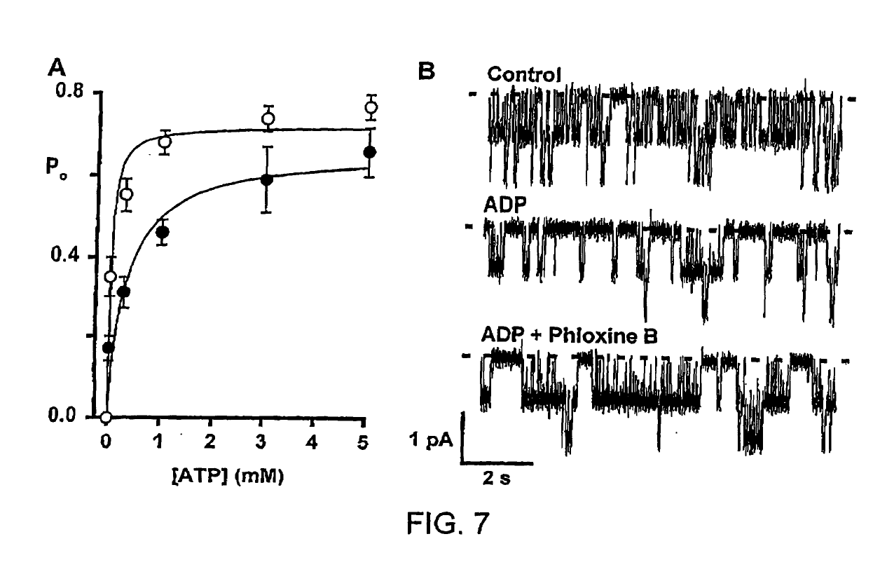

FIG. 7: Phloxine B (1 μM) Enhances ATP-Dependent Stimulation and Relieves ADP Inhibition of CFTR

A, relationship between ATP concentration and Pₒ in the absence (filled symbols) and presence (open symbols) of phloxine B (1 μM). Symbols and error bars are means±SEM of n=5-15 observations at each ATP concentration. Continuous lines are Michaelis-Menton fits to the mean data. B, representative recordings show the effect of phloxine B (1 μM) on the activity of two CFTR Cl⁻¹ channel inhibited by ADP (0.3 mM). ATP (0.3 mM) and PKA (75 nM) were continuously present in the intracellular solution. Other details as in FIG. 5A. Phloxine B (1 μM) relieved ADP (0.3 mM) Inhibition of Pₒ (control, Pₒ=0.43±0.03; ADP, Pₒ=0.21±0.02; ADP+phloxine B, Pₒ=0.43±0.05; n=5). ADP (0.3 mM) decreased Pₒ to 49±1% of the control value (P<0.001). However, in the presence of phloxine B (1 μM), ADP (0.3 mM) was without effect on Pₒ (P>0.05).

FIG. 7A shows that as the ATP concentration increased, Pₒ values in both the absence and presence of phloxine B (1 μM) increased. However, at each concentration of ATP tested, Pₒ values in the presence of phloxine B (1 μM) were greater than those recorded in the absence of the drug (FIG. 7A). In both the absence and presence of phloxine B (1 μM) the relationship between Pₒ and ATP concentration was best fitted by a Michaelis-Menten function (control: Kₘ=331 μM, maximum Pₒ (Pₒₘₐₓ)=0.66; phloxine B; Kₘ=39 μM, Pₒₘₐₓ=0.72). These data suggest that phloxine B (1 μM) increases the affinity of CFTR for ATP. They also suggest that phloxine B and ATP might not compete with ATP for a common binding site.

Anderson and Welsh (19) demonstrated that mutations in NBD2 suppress ADP inhibition of CFTR Cl⁻ channels. To examine the effect of phloxine B (1 μM) on ADP inhibition of CFTR Cl⁻ channels, we first added ADP (0.3 mM) and then added phloxine B (1 μM) to the intracellular solution in the continuous presence of ATP and PKA (FIG. 7B). FIG. 7B shows that phloxine B (1 μM) relieved ADP inhibition of CFTR. However, visual inspection of the data suggests that phloxine B (1 μM) did not prevent the ADP-induced increase in the interbust interval. Instead, phloxine B (1 μM) prolonged the duration of bursts of channel activity.

Therapeutic Potential of Phloxine B for the Treatment of CF

To begin to evaluate the therapeutic potential of phloxine B, we investigated whether phloxine B (1 μM) stimulates the activity of CF-associated mutants. The most common CF-associated mutation ΔF508 disrupts both the biosynthesis and function of CFTR (13). To investigate whether phloxine B stimulates the activity of ΔF508 CFTR Cl⁻ channels, we grew cells at 28° C. to overcome the defective processing of ΔF508 and facilitate its delivery to the cell membrane.

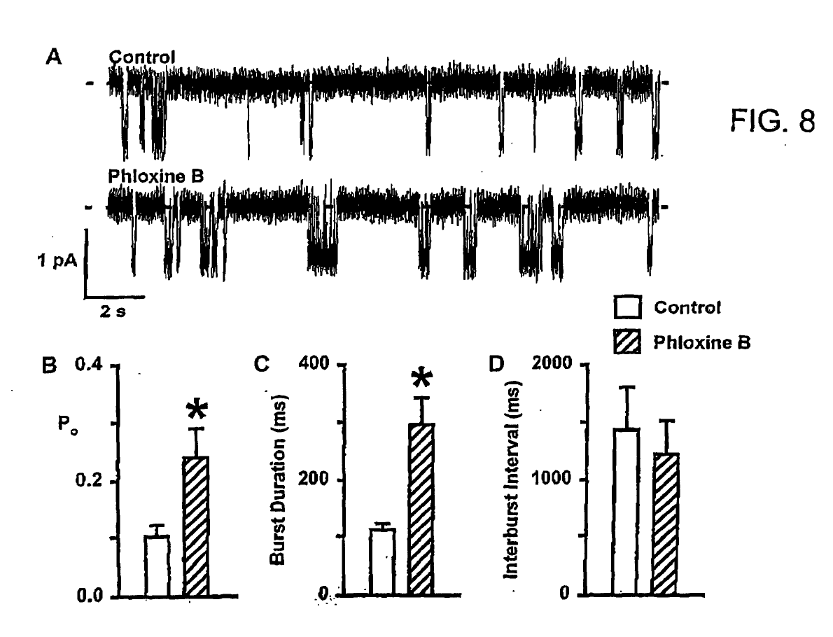

FIG. 8. Phloxine B (1 μM) Stimulates the Activity of the CF-Associated Mutant ΔF508

A, representative recordings show the effect of phloxine B (1 μM) on the activity of a single ΔF508 CFTR Cl⁻ channel. ATP (1.0 mM) and PKA (75 nM) were continuously present in the intracellular solution. Other details as in FIG. 5A. B, C and D, effect of phloxine B on Pₒ, burst duration and interburst interval. Columns and error bars indicate means±SEM; n=3-5 observations at each concentration. The asterisks indicate values that are significantly different from the control value (P<0.05). Other details as in A.

FIG. 8 shows the pattern of gating of a single ΔF508 CFTR Cl⁻ channel following phosphorylation by PKA and the effect of phloxine B (1 μM) on ΔF508 gating behaviour. Although the mean burst duration of ΔF508 CFTR Cl⁻ channels is similar to that of wild-type CFTR, ΔF508 CFTR Cl⁻ channels have a greatly prolonged interburst interval (FIGS. 6 and 8). As a result, the Pₒ of ΔF508 is significantly less than that of wild-type CFTR (FIGS. 5 and 8). Phloxine B (1 μM) increased the Pₒ of ΔF508 1.5-fold by dramatically prolonging the mean burst duration of ΔF508 CFTR Cl channels (P<0.05) without altering the interburst interval (P>0.05; FIG. 8). These data demonstrate that phloxine B (1 μM) is a potent activator of ΔF508 CFTR Cl⁻ channels. They also suggest that the mechanism of phloxine B stimulation of wild-type and ΔF508 CFTR Cl⁻ channels is the same.

To learn whether phloxine B stimulates CFTR in intact cells and to test whether the drug activates native CFTR Cl⁻ channels, we investigated the effect of phloxine B on cAMP-stimulated iodide efflux from Type I MDCK cells. Type I MDCK cells are kidney epithelial cells that endogenously express the CFTR Cl⁻ channel (20).

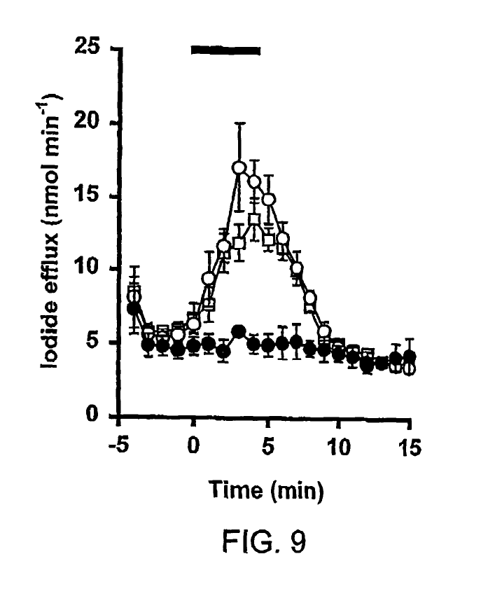

FIG. 9: Phloxine B Enhances cAMP-Stimulated Iodide Efflux from Type I MDCK Cells

Data show the time course of iodide efflux from Type I MDCK cells. Nitrate was substituted for iodide in the bathing medium at t=−4 min and iodide efflux was monitored at 1 min intervals, for the duration of the experiment. The bar indicates the presence of the test drugs (cAMP agonists (forskolin (10 μM), 3-isobutyl-1-methylxanthine (IBMX; 100 μM), and 8-(4-chlorophenylthio) adenosine 3′:5′-cyclic monophosphate (CPT-cAMP; 500 μM)), open square; phloxine B (10 μM), filled circle; cAMP agonists and phloxine B (10 μM), open circle). Symbols and error bars are means±SEM (phloxine B, n=4; cAMP agonists and cAMP agonists+phloxine B, n=8) values at each time point.

Under control conditions, cAMP agonists stimulated an efflux of iodide with a peak response of 13.6±1.5 nmol min⁻¹ (n=8) occurring 5 min after agonist addition (FIG. 9). Phloxine B (10 μM) enhanced both the speed and magnitude (P<0.05) of the cAMP-stimulated iodide efflux (FIG. 9). By contrast, in the absence of cAMP agonists the drug was without effect. These data demonstrate that phloxine B stimulates endogenous CFTR Cl⁻ channels in intact cells.

Discussion

Our data indicate that fluorescein derivatives, such as phloxine B, modulate the activity of the CFTR Cl⁻ channel in two ways. First, low micromolar concentrations of these drugs stimulate channel activity. Second, at elevated concentrations they inhibit the CFTR Cl⁻ channel. Our data also suggest that phloxine B potently stimulates the activity of the most common CF-associated mutant, ΔF508 and activates native CFTR Cl⁻ channels in intact epithelial cells.

Previous work has identified several pharmacological strategies to manipulate the activity of the CFTR Cl⁻ channel. Some agents modulate the activity of the protein kinases and phosphatases that regulate CFTR (2,21), whereas other agents interact directly with CFTR to control channel activity (8,21). The CFTR activator that has received the most attention is the flavonoid genistein. Genistein interacts directly with NBD2 to prolong the lifetime of the open channel conformation (18,22,23). Our data indicate that the mechanism of phloxine B stimulation of CFTR shows some similarities to that of genistein. First, the activation of CFTR by both genistein and phloxine B required the prior phosphorylation of CFTR by PKA (18, present study). Second both genistein and phloxine B enhanced the activity of PKA-phosphorylated CFTR Cl⁻ channels in excised inside-out membrane patches (18,22 and present study). Third, both genistein and phloxine B stimulated phosphorylated CFTR Cl⁻ channels by greatly prolonging the duration of channel openings (18,22 and present study). Based on these data, we propose that phloxine B interacts directly with NBD2 to prolong the lifetime of the open channel conformation.

The mechanism of phloxine B stimulation of CFTR also shows some differences from that of genistein. First, Wang et al. (18) demonstrated that the effect of genistein on channel activity depends on the phosphorylation status of CFTR. The authors showed that genistein enhanced the activity of weakly phosphorylated CFTR Cl⁻ channels, but had little or no effect on the activity of strongly phosphorylated CFTR Cl⁻ channels. In contrast, phloxine B stimulation of CFTR Cl⁻ channels was not dependent on the Pₒ of CFTR (n=10, data not shown), This suggests that like the inorganic phosphate analogues orthovanadate, beryllium fluoride and pyrophosphate (2,3), the effect of phloxine B on channel activity does not depend on the phosphorylation status of CFTR. Second, increasing ATP concentrations did not affect genistein stimulation of CFTR (18,24). In contrast, phloxine B altered the relationship between Pₒ and ATP concentration. The change in Kₘ suggests that phloxine B might enhance ATP binding, hydrolysis or a conformation change following ATP hydrolysis. Moreover, the increased Pₒₘₐₓ and the effect of phloxine B on ADP inhibition of CFTR suggest that phloxine B might alter the rate of ATP hydrolysis or a post-hydrolytic event. Finally, our data suggest that fluorescein derivatives are more potent activators of CFTR than genistein. Wang et al. (18) reported that genistein (35 μM) maximally stimulated the active of wild-type CFTR Cl⁻ channels, whereas we found that phloxine B (5 μM) and bengal rose B (1 μM) maximally stimulated channel activity in excised membrane patches. Thus, our data indicate that fluorescein derivatives are potent activators of the CFTR Cl⁻ channel.

The observation that phloxine B stimulates CFTR Cl⁻ channels has implications for the treatment of diseases associated with diminished CFTR function. Some CF-associated mutants, Including the most common, ΔF508, disrupt the processing of CFTR and its delivery to the cell membrane (4). In contrast other mutants, including R347P and G551D that are processed normally and delivered to the cell membrane, disrupt the properties and regulation of the CFTR Cl⁻ channel (4). This suggests that patients bearing mutant Cl⁻ channels present at the cell membrane might be treated with drugs, which enhance the activity of the CFTR Cl⁻ channel.

For activators of the CFTR Cl⁻ channel to be of value as a treatment for CF, two important criteria must be fulfilled. First, they must activate mutant CFTR Cl⁻ channels. Second, unless they interact with plasma membrane receptors to increase intracellular levels of cAMP, they must be delivered to the interior of epithelial cells. Our data indicate that phloxine B (1 μM) potently stimulates the activity of ΔF508 CFTR Cl⁻0 channels when the mutant protein is present at the cell membrane. This satisfies the first criteria. Fluorescein derivatives are lipophilic anions, suggesting that they will gain access to the interior of epithelial cells by permeating through the lipid phase of the cell membrane by non-ionic diffusion. Consistent with this idea, de Weille et al. (10) showed that phloxine B inhibits K_{ATP} currents in pancreatic β-cells when added to the extracellular solution. Moreover, we found that addition of phloxine B (10 μM) to the extracellular solution enhances cAMP-stimulated iodide efflux from MDCK Type I cells that endogenously express wild-type CFTR. These data satisfy the second criteria. Thus, phloxine B and related fluorescein compounds, especially bengal rose B the most potent CFTR activator that we have identified, have valuable therapeutic properties.

### REFERENCES

1. Riordan, J. R., J. M. Rommens, B.-S. Kerem, N. Alon, R. Rozmahel, Z. Grzelczak, J. Zielenski, S. Lok, N. Plavsic, J.-L. Chou, M. L. Drumm, M. C. Iannuzzi, F. S. Collins, and L. Tsui. 1989. Identification of the cystic fibrosis gene: cloning and characterization of complementary DNA. Science 245:1066-1073.

2. Gadsby, D. C. and A. C. Naim. 1999. Control of cystic fibrosis transmembrane conductance regulator channel gating by phosphorylation and nucleotide hydrolysis. Physiological Reviews 79 (Supplement 1):S77-S107.

3. Sheppard, D. N. and M. J. Welsh. 1999. Structure and function of the cystic fibrosis transmembrane conductance regulator chloride channel. Physiological Reviews 79 (Supplement 1):S23-S45.

4. Welsh, M. J., L. Tsui, T. F. Boat, and A. L. Beaudet. 1995. Cystic fibrosis. In The Metabolic and Molecular Basis of Inherited Disease. C. R. Scriver, A. L. Beaudet, W. S., Sly, and D. Valle, editors. McGraw-Hill Inc. New York. 3799-3876.

5. Sullivan, L. P., D. P. Wallare, and J. J. Grantham. 1998. Epithelial transport in polycystic kidney disease. Physiological Reviews 78:1165-1191.

6. Gabriel, S. E., K N. Brigman, B. H. Koller, R. C. Boucher, and M. J. Stutts. 1994. Cystic fibrosis heterozygote resistance to cholera toxin in the cystic fibrosis mouse model. Science 266:107-109.

7. Welsh, M. J. and A. E. Smith. 1993. Molecular mechanisms of CFTR chloride channel dysfunction in cystic fibrosis. Cell 73:1251-1254.

8. Hwang, T.-C. and D. N. Sheppard. 1999. Molecular pharmacology of the CFTR Cl⁻ channel. Trends in Pharmacological Sciences 20:448-453.

9. Ashcroft, F. M. and F, M. Gribble. 1998. Correlating structure and function in ATP-sensitive K⁺ channels. Trends in Neurosciences 21:288-294.

10. de Weille, J. R., M. Müller, and M. Lazdunski. 1992. Activation and inhibition of ATP-sensitive K⁺ channels by fluorescein derivatives. Journal of Biological Chemistry 267:4557-4563.

11. Marshall, J., S. Fang, L, S. Ostedgaard. C. R. O'Riordan, D. Ferrara, J. F. Amara, H. Hoppe IV, R. K. Scheule, M. J. Welsh, A. E. Smith, and S. H. Cheng. 1994. Stoichiometry of recombinant cystic fibrosis transmembrane conductance regulator in epithelial cells and its functional reconstitution into cells in vitro. Journal of Biological Chemistry 269:2987-2995.

12. Sheppard, D. N. and K. A. Robinson. 1997. Mechanism of glibenclamide inhibition of cystic fibrosis transmembrane conductance regulator Cl⁻ channels expressed in a murine cell line. Journal of Physiology 503:333-346.

13. Denning, G. M., M. P. Anderson, J. F. Amara, J. Marshall, A. E. Smith, and M. J. Welsh. 1992. Processing of mutant cystic fibrosis transmembrane conductance regulator is temperature-sensitive. Nature 358:761-764.

14. Hamill, O. P., A. Marty, E. Neher, B. Sakmann, and F. J. Sigworth. 1981. Improved patch-clamp techniques for high-resolution current recording from cells and cell-free membrane patches. Pflügers Archiv 391:85-100.

15. Carson, M. R., S. M. Travis, and M. J. Welsh. 1995. The two nucleotide-binding domains of cystic fibrosis transmembrane conductance regulator (CFTR) have distinct functions in controlling channel activity. Journal of Biological Chemistry 270:1711-1717.

16. Lansdell, K. A., J. F. Kidd, S. J. Delaney, B. J. Wainwright, and D. N. Sheppard. 1998. Regulation of murine cystic fibrosis transmembrane conductance regulator Cl⁻ channels expressed in Chinese hamster ovary cells. Journal of Physiology 512:751-764.

17. Hwang, T.-C., G. Nagel, A. C. Naim, and D.C. Gadsby. 1994. Regulation of the gating of cystic fibrosis transmembrane conductance regulator Cl channels by phosphorylation and ATP hydrolysis. Proceedings of the National Academy of Sciences USA 91:4698-4702.

18. Wang, F., S. Zeltwanger, I. C.-H. Yang, A. C. Naim, and T.-C. Hwang. 1998. Actions of genistein on cystic fibrosis transmembrane conductance regulator channel gating: evidence for two binding sites with opposite effects. Journal of General Physiology 111:477-490.

19. Anderson, M. P. and M. J. Welsh. 1992. Regulation by ATP and ADP of CFTR chloride channels that contain mutant nucleotide-binding domains. Science 257:1701-1704.

20. Mohamed, A., D. Ferguson, F. S. Seibert, H.-M. Cai, N. Kartner, S. Grinstein, J. R. Riordan, and G. L. Lukacs. 1997. Functional expression and apical localization of the cystic fibrosis transmembrane conductance regulator in MDCK I cells, Biochemical Journal 322:259-265.

21. Schultz B. D., A. K. Singh, D. C. Devor, and R. J, Bridges 1999. Pharmacology of CFTR chloride channel activity. Physiological Reviews 79 (Supplement 1):S109-S144.

22. French, P. J., J. Bljman, A. G. Bot, W. E. M. Boomaars, B. J. Scholte, and H. R. de Jonge. 1997. Genistein activates CFTR Cl⁻ channels via a tyrosine kinase- and protein phosphatase-independent mechanism. American Journal of Physiology 273:C747-C753.

23. Randak. C., E. A. Auerswald, I. Assfalg-Machleidt, W. W. Reenstra, and W. Machleidt. 1999. Inhibition of ATPase, GTPase and adenylate kinase activities of the second nucleotide binding fold of cystic fibrosis transmembrane conductance regulator by genistein. Biochemical Journal 340:227-235.

24. Weinreich, F., P. G. Wood, J. R. Riordan, and G. Nagel. 1997. Direct action of genistein on CFTR. Pflügers Archiv 434:484-491.

1. The use of a compound of the formula I 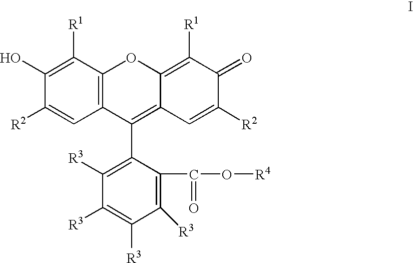
wherein R¹, R² and R³ may each be the same or a different group selected from H, 1 to 6C alkyl and halo and R⁴ is a group selected from H and 1 to 6C alkyl, and pharmaceutically-acceptable salts thereof, in the manufacture of a medicament for the treatment of a disease or medical condition of a living animal body, including a human, which disease or condition is responsive to the activation of the cystic fibrosis transmembrane conductance regulator chloride channels.

2. The use according to claim 1, wherein the compound has the formula I in which R¹, R² and R³ is the same or different and is selected from H, Cl, Br and I and R⁴ is selected from H and 1 to 4C alkyl, and pharmaceutically-acceptable salts thereof.

3. The use according to claim 2, wherein the compound has the formula I in which R¹ is selected from H, Cl, Br and I; Re is selected from H, Cl, Br and I; R³ is select d from H, Cl, Br and I; and R⁴ Is selected from H, methyl and ethyl.

4. The use according to claim 3, wherein the compound has the formula I in which R¹ and R² are both Br, R³ is Cl and R⁴ is H.

5. The use according to claim 3, wherein the compound has the formula I in which R¹ and R² are both I, R³ is Cl and R⁴ is H.

6. The use according to claim 3, wherein the compound has the formula I in which R¹ and R² are both Br and R³ and R⁴ are both H.

7. The use according to any one of claims 1 to 6, wherein the disease responsive to the activation of the cystic fibrosis transmembrane conductance regulator chloride channel is cystic fibrosis.

8. The use according to any one of claims 1 to 6, wherein the disease or condition responsive to the activation of the cystic fibrosis transmembrane conductance regulator chloride channel is selected from disseminated bronchiectasis, pulmonary infections, chronic pancreatitis and some forms of male infertility.

9. The use according to any one of claims 1 to 6, wherein the disease or condition responsive to activation of the cystic fibrosis transmembrane conductance regulator chloride channel are the inherited and acquired forms of the cardiac disease long QT, syndrome.

10. A method of treating a disease or medical condition of a living animal body, including a human, which disease or condition is responsive to the activation of the cystic fibrosis transmembrane conductance regulator (CFTR) chloride channels which method comprises administering to the living animal body a CFTR chloride channel activating amount of a compound of the formula I 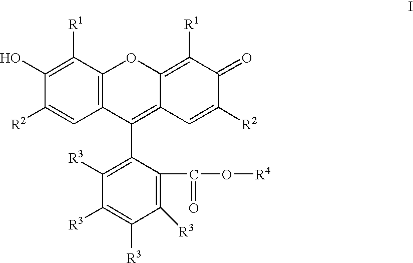
wherein each of R¹, R² and R³, which may be the same or different, is selected from H, 1 to 6C alkyl and halo and R⁴ is a group selected from H and 1 to 6C alkyl, and pharmaceutical-acceptable salts thereof.

11. A method according to claim 10, wherein the compound has the formula I in which R¹, R² and R³ is the same or different and is selected from H, Cl, Br and I and R⁴ is selected from H and 1 to 4C alkyl, and pharmaceutically-acceptable salts.

12. A method according to claim 11, wherein the compound has the formula I in which R¹ is selected from H, Cl, Br and I; R² is selected from H, Cl, Br and I; R³ is selected from H, Cl, Br and I and R⁴ is selected from H, methyl and ethyl.

13. A method according to claim 12, wherein the compound has the formula I in which R¹ and R² are both Br, R³ is Cl and R⁴ is H.

14. A method according to claim 12, wherein the compound has the formula I in which R¹ and R² are both I, R³ is Cl and R⁴ is H.

15. A method according to claim 12, wherein the compound has the formula I in which R¹ and R² are both Br and R³ and R⁴ are both H.

16. A method according to any one of claims 10 to 15 wherein the disease to be treated is cystic fibrosis.

17. A method according to any one of claims 10 to 15, wherein the disease or condition to be treated is selected from disseminated bronchiectasis, pulmonary infections, chronic pancreatitis and some forms of male infertility.

18. A method according to any one of claims 10 to 15, wherein the disease or condition to be treated is selected from the inherited and acquired forms of long QT syndrome.

19. The use according to any one of claims 1 to 9, to enhance the activity of CFTR in patent cells corrected by gene therapy, protein therapy, and treatments that deliver misprocessed CFTR to the apical membrane.
